(19) United States
(12) Patent Application Publication
(10) Pub. No.: US 2017/0365790 A1
(43) Pub. Date: Dec. 21, 2017

(54) CARBAZOLE DERIVATIVE AND ORGANIC ELECTROLUMINESCENT DEVICE

(71) Applicant: Industrial Technology Research Institute, Hsinchu (TW)

(72) Inventors: Po-Sheng Wang, Taichung City (TW); Jiun-Haw Lee, Taipei City (TW); Man-Kit Leung, Taipei City (TW); Hsin-Jen Chen, Tainan City (TW); Yu-Hsuan Hsieh, Taipei City (TW); Chi-Feng Lin, New Taipei City (TW); Tien-Lung Chiu, Taipei City (TW)

(73) Assignee: Industrial Technology Research Institute, Hsinchu (TW)

(21) Appl. No.: 15/696,193

(22) Filed: Sep. 6, 2017

Related U.S. Application Data
(63) Continuation of application No. 14/309,900, filed on Jun. 20, 2014.

(30) Foreign Application Priority Data
Feb. 25, 2014 (TW) 103106222

(51) Int. Cl. H01L 51/00; C07D 413/10; C07D 403/10; C09K 11/06; C07D 403/14; C07D 413/14; H01L 51/50

(52) U.S. Cl. CPC H01L 51/0067; H01L 51/0069; C07D 403/14; C07D 413/10; C07D 413/14; H01L 51/0072; H01L 51/007; C07D 403/10; C09K 11/06; C09K 2211/185; H01L 51/0085; H01L 51/50; H01L 51/5016

(57) ABSTRACT

A carbazole derivative is provided. The carbazole derivative is shown in formula (9):

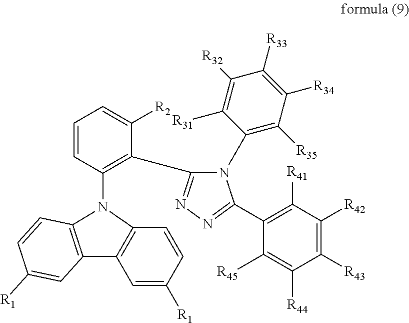

### CROSS-REFERENCE TO RELATED APPLICATION

This application is a continuation application of and claims the priority benefit of a prior application Ser. No. 14/309,900, filed on Jun. 20, 2014, now pending. The prior application Ser. No. 14/309,900 claims the priority benefit of Taiwan application serial no. 103106222, filed on Feb. 25, 2014. The entirety of each of the above-mentioned patent applications is hereby incorporated by reference herein and made a part of this specification.

### TECHNICAL FIELD

The disclosure relates to a carbazole derivative and an organic electroluminescent device containing the same.

### BACKGROUND

An electroluminescent device is a semiconductor device capable of converting electrical energy into optical energy and having a high conversion efficiency. The electroluminescent device is wildly used as, for instance, a luminescent element in an indicator light, a display panel, and an optical reading/writing head. Since the electroluminescent device has properties such as wide viewing angle, simple processing, low production cost, fast response, wide operation temperature range, and full color display, the electroluminescent device can be expected to become the mainstream of the next generation of flat panel display.

In general, the organic electroluminescent device includes an anode, an organic luminescent layer, and a cathode, wherein the organic luminescent layer includes a host material and a guest material. The holes and the electrons in the organic electroluminescent device are mainly transported to the host material to be combined and thereby generate energy, and then the energy is transferred to the guest material to generate light. Therefore, the host material needs to have good electron and hole transport properties, and the triplet state energy level thereof needs to be higher or equal to the triplet state energy level of the guest material to prevent energy loss due to reverse energy transfer.

In addition to selecting the material of the organic luminescent layer based on energy level, the material also needs to have good thin film stability and a high glass transition temperature (T_{g}). The current red and green phosphorescent light-emitting diodes generally have good service life and performance. However, the triplet state energy level of the guest material of the blue phosphorescent light-emitting diode is higher than the triplet state energy level of the red and green guest materials, and therefore the blue phosphorescent light-emitting diode needs a host material having a higher triplet state energy level.

To increase the triplet state energy level of the host material, the conjugation length in the molecules of the host material needs to be reduced. However, reducing the conjugation length in the molecules of the host material reduces the molecular weight thereof, and the smaller the molecular weight of the host material, the lower the thermal stability (indicated by glass transition temperature) of the host material. To solve the issue of thermal stability of the host material, the research of introducing a large group substituent (such as SimCP or CzSi) to an N,N′-dicarbazolyl-3,5-benzene (mCP) molecule was done to increase the glass transition temperature of the molecule without affecting the conjugation length of the molecule. However, the large group substituent may damage the stacking between host material molecules, such that the transport distance of a carrier jumping between the host material molecules is longer. As a result, the carrier transport properties of the host material are lowered. Therefore, a host material capable of meeting the requirements of a high triplet state energy level, bipolar carrier transport properties, and thermal stability at the same time is urgently needed.

### SUMMARY

The disclosure provides a carbazole derivative.

The disclosure provides an organic electroluminescent device. The organic electroluminescent device includes an organic luminescent material containing the carbazole derivative.

The carbazole derivative of the disclosure is shown in formula (1):

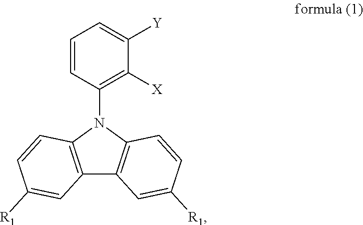

wherein X can represent one of the groups shown in formula (2) to formula (3):

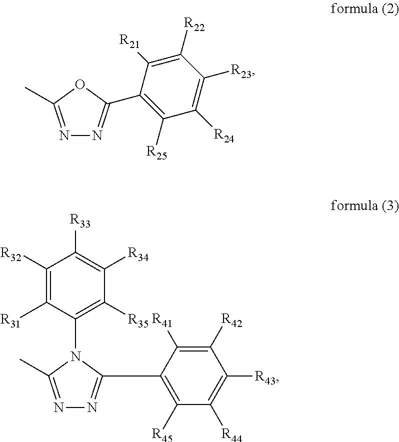

Y can represent R₂ or a group shown in formula (4):

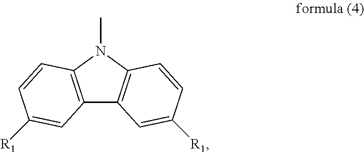

and

R₁, R₂, R₂₁, R₂₂, R₂₃, R₂₄, R₂₅, R₃₁, R₃₂, R₃₃, R₃₄, R₃₅, R₄₁, R₄₂, R₄₃, and R₄₄ can independently be selected from one of a hydrogen atom, a fluorine atom, a cyano group, a substituted or non-substituted straight-chain or branched-chain alkyl group, a substituted or non-substituted cycloalkyl group, a substituted or non-substituted straight-chain or branched-chain alkoxy group, a substituted or non-substituted straight-chain or branched-chain thioalkyl group, and a substituted or non-substituted straight-chain or branched-chain alkenyl group; and R₄₅ can independently be selected from one of a hydrogen atom, a fluorine atom, a cyano group, a substituted or non-substituted straight-chain or branched-chain alkyl group, a substituted or non-substituted cycloalkyl group, a substituted or non-substituted straight-chain or branched-chain alkoxy group, a substituted or non-substituted straight-chain or branched-chain thioalkyl group, a substituted or non-substituted straight-chain or branched-chain alkenyl group, and the group shown in formula (5):

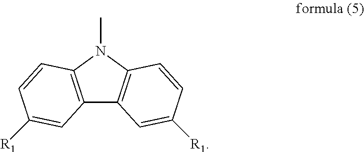

An organic electroluminescent device of the disclosure can include a first electrode layer, a second electrode layer, and an organic luminescent unit. The organic luminescent unit is located between the first electrode layer and the second electrode layer. The organic luminescent unit includes a carbazole derivative shown in formula (1):

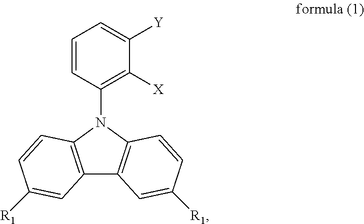

wherein X can represent one of the groups shown in formula (2) to formula (3):

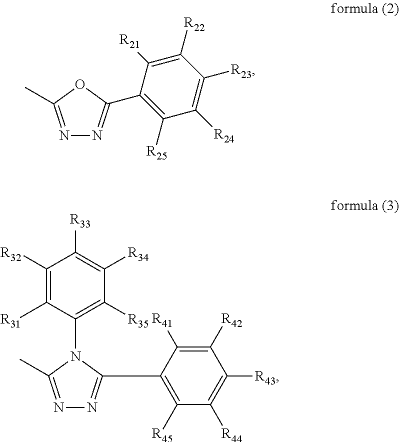

Y can represent R₂ or a group shown in formula (4):

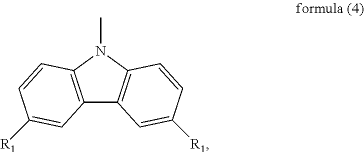

and

R₁, R₂, R₂₁, R₂₂, R₂₃, R₂₄, R₂₅, R₃₁, R₃₂, R₃₃, R₃₄, R₃₅, R₄₁, R₄₂, R₄₃, and R₄₄ can independently be selected from one of a hydrogen atom, a fluorine atom, a cyano group, a substituted or non-substituted straight-chain or branched-chain alkyl group, a substituted or non-substituted cycloalkyl group, a substituted or non-substituted straight-chain or branched-chain alkoxy group, a substituted or non-substituted straight-chain or branched-chain thioalkyl group, and a substituted or non-substituted straight-chain or branched-chain alkenyl group; and R₄₅ can independently be selected from one of a hydrogen atom, a fluorine atom, a cyano group, a substituted or non-substituted straight-chain or branched-chain alkyl group, a substituted or non-substituted cycloalkyl group, a substituted or non-substituted straight-chain or branched-chain alkoxy group, a substituted or non-substituted straight-chain or branched-chain thioalkyl group, a substituted or non-substituted straight-chain or branched-chain alkenyl group, and the group shown in formula (5):

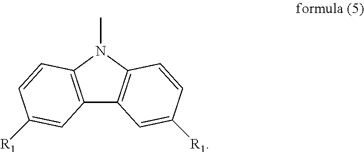

Another organic electroluminescent device of the disclosure can include a first electrode layer, a second electrode layer, and an organic luminescent unit. The organic luminescent unit is located between the first electrode layer and the second electrode layer. The organic luminescent unit includes an organic luminescent layer. The organic luminescent layer includes a host material and a guest material. The host material includes a carbazole derivative shown in formula (1):

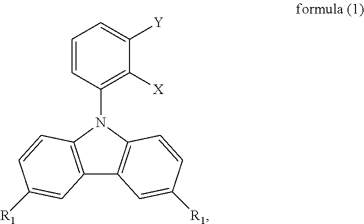

wherein X can represent one of the groups shown in formula (2) to formula (3):

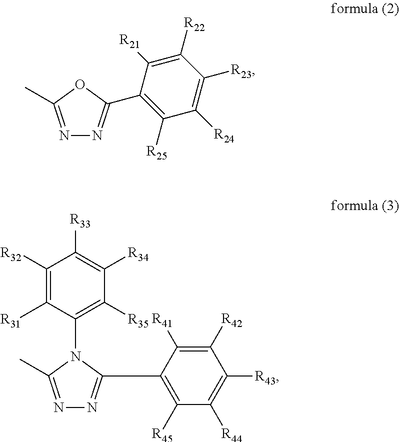

Y can represent R₂ or a group shown in formula (4):

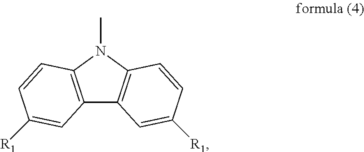

and

R₁, R₂, R₂₁, R₂₂, R₂₃, R₂₄, R₂₅, R₃₁, R₃₂, R₃₃, R₃₄, R₃₅, R₄₁, R₄₂, R₄₃, and R₄₄ can independently be selected from one of a hydrogen atom, a fluorine atom, a cyano group, a substituted or non-substituted straight-chain or branched-chain alkyl group, a substituted or non-substituted cycloalkyl group, a substituted or non-substituted straight-chain or branched-chain alkoxy group, a substituted or non-substituted straight-chain or branched-chain thioalkyl group, and a substituted or non-substituted straight-chain or branched-chain alkenyl group; and R₄₅ can independently be selected from one of a hydrogen atom, a fluorine atom, a cyano group, a substituted or non-substituted straight-chain or branched-chain alkyl group, a substituted or non-substituted cycloalkyl group, a substituted or non-substituted straight-chain or branched-chain alkoxy group, a substituted or non-substituted straight-chain or branched-chain thioalkyl group, a substituted or non-substituted straight-chain or branched-chain alkenyl group, and the group shown in formula (5):

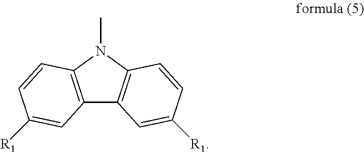

In order to make the aforementioned features and advantages of the disclosure more comprehensible, embodiments accompanied with figures are described in detail below.

### BRIEF DESCRIPTION OF THE DRAWINGS

The accompanying drawings are included to provide a further understanding of the disclosure, and are incorporated in and constitute a part of this specification. The drawings illustrate embodiments of the disclosure and, together with the description, serve to explain the principles of the disclosure.

FIG. 1 is a cross-sectional schematic diagram of an organic electroluminescent device of an embodiment of the disclosure.

FIG. 2 is a cross-sectional schematic diagram of an organic electroluminescent device of another embodiment of the disclosure.

FIG. 3 is a cross-sectional schematic diagram of an organic electroluminescent device of yet another embodiment of the disclosure.

### DETAILED DESCRIPTION OF DISCLOSED EMBODIMENTS

### [Organic Luminescent Material]

The following description is of the best-contemplated mode of carrying out the disclosure. This description is made for the purpose of illustrating the general principles of the disclosure and should not be taken in a limiting sense. The scope of the disclosure is best determined by reference to the appended claims.

An organic luminescent material of the disclosure includes a host material and a guest material. The host material can include a carbazole derivative shown in formula (1):

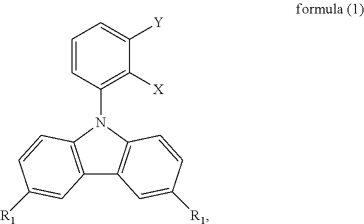

wherein X can represent one of the groups shown in formula (2) to formula (3):

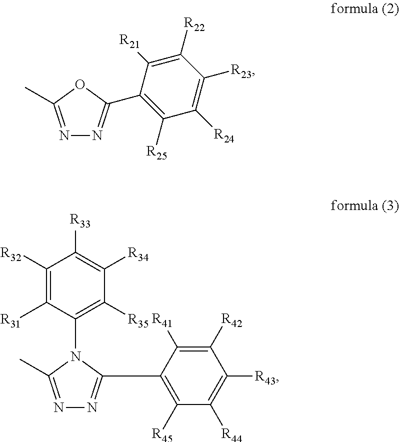

Y can represent R₂ or a group shown in formula (4):

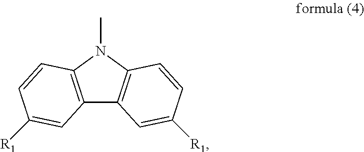

and

R₁, R₂, R₂₁, R₂₂, R₂₃, R₂₄, R₂₅, R₃₁, R₃₂, R₃₃, R₃₄, R₃₅, R₄₁, R₄₂, R₄₃, and R₄₄ can independently be selected from one of a hydrogen atom, a fluorine atom, a cyano group, a substituted or non-substituted straight-chain or branched-chain alkyl group, a substituted or non-substituted cycloalkyl group, a substituted or non-substituted straight-chain or branched-chain alkoxy group, a substituted or non-substituted straight-chain or branched-chain thioalkyl group, and a substituted or non-substituted straight-chain or branched-chain alkenyl group; and R₄₅ can independently be selected from one of a hydrogen atom, a fluorine atom, a cyano group, a substituted or non-substituted straight-chain or branched-chain alkyl group, a substituted or non-substituted cycloalkyl group, a substituted or non-substituted straight-chain or branched-chain alkoxy group, a substituted or non-substituted straight-chain or branched-chain thioalkyl group, a substituted or non-substituted straight-chain or branched-chain alkenyl group, and the group shown in formula (5):

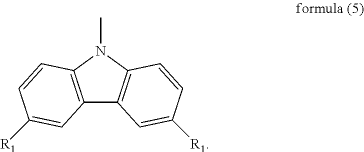

Several embodiments of the carbazole derivatives is shown in formula (6) to formula (10):

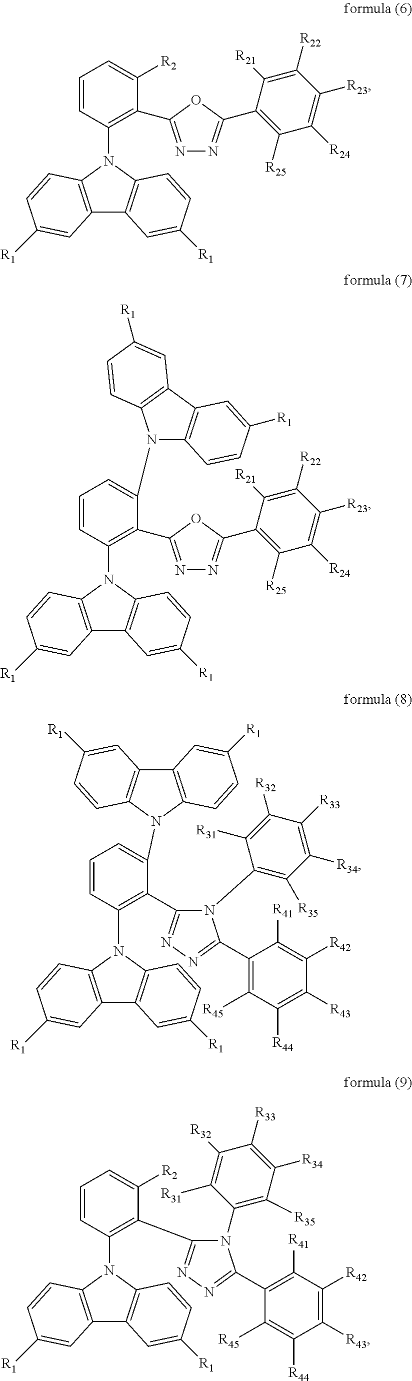

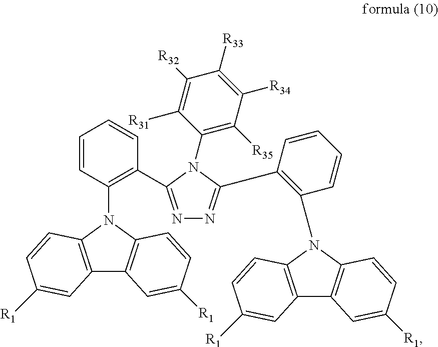

wherein,

R₁, R₂, R₂₁, R₂₂, R₂₃, R₂₄, R₂₅, R₃₁, R₃₂, R₃₃, R₃₄, R₃₅, R₄₁, R₄₂, R₄₃, R₄₄, and R₄₅ are as defined above.

Several embodiments of the carbazole derivatives is embodiment shown in formula (11) to formula (15):

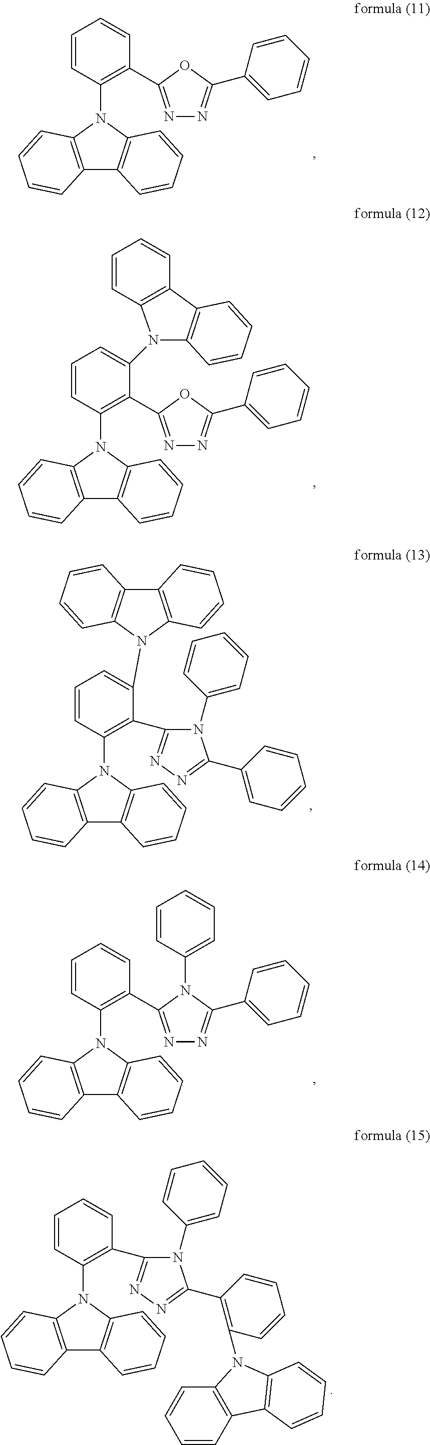

In the embodiment, the host material including the carbazole derivative shown in formula (1) has an electron-accepting group and an electron-donating group. Specifically, the carbazole group is an electron-donating group having an effect of pushing electrons and can be used to transport holes. The groups shown in formula (2) and formula (3) are electron-accepting groups having an effect of pulling electrons and can be used to transport electrons. In other words, the host material of the embodiment can have both an electron-accepting group and an electron-donating group in the same molecule to achieve properties of a bipolar carrier transport.

It should be mentioned that, to improve the luminous efficiency of the organic luminous layer, the triplet state energy level of the host material needs to be higher or equal to the triplet state energy level of the guest material to prevent a reduction in luminous efficiency of the luminescent device caused by reverse energy transfer. In the embodiment, as shown in formula (1), the benzene ring of formula (1) can be connected to the group of formula (2) or formula (3) at an ortho position (i.e., X position) connected to a carbazole group. In this way, the steric effect formed by the group of formula (2) or formula (3) and the carbazole group can cause a twist to the carbazole derivative of formula (1) such that the conjugation length of the carbazole derivative is not increased. Therefore, the host material including the carbazole derivative of formula (1) of the embodiment can have a high triplet state energy level, thereby preventing the effect of reverse energy transfer. As a result, the luminous efficiency of the organic electroluminescent device can be increased.

Moreover, the guest material of the embodiment can be any material suitable for the organic luminescent layer of the organic electroluminescent device, and is, for instance, one of the compounds shown in formula (16) (i.e., the known Ir(2-phq)₃), formula (17) (i.e., the known Ir(ppy)₃), and formula 18 (i.e., the known Flrpic). However, the disclosure is not limited thereto.

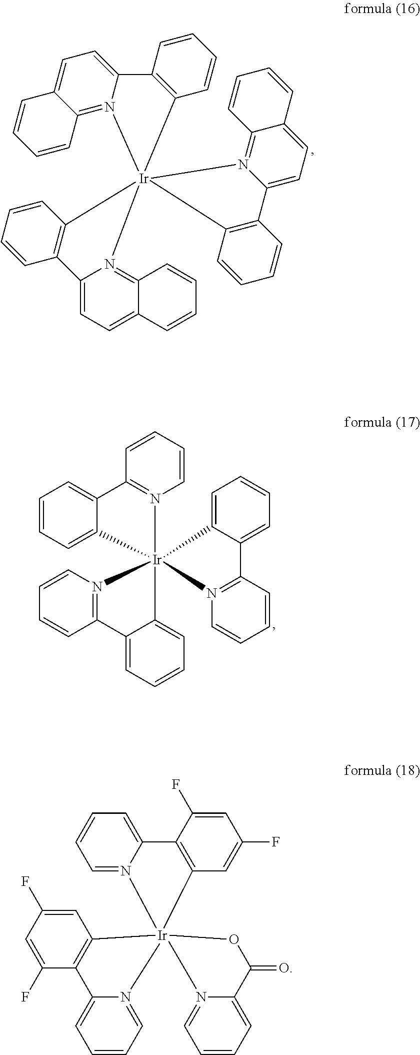

It should be mentioned that, the material of the disclosure including the carbazole derivative shown in formula (1) is not only suitable for the host material of the organic luminescent layer, but is also suitable for each of the film layers in the organic luminescent unit such as a hole injection layer, a hole transport layer, an electron blocking layer, an electron injection layer, or an electron transport layer.

### [Organic Electroluminescent Device]

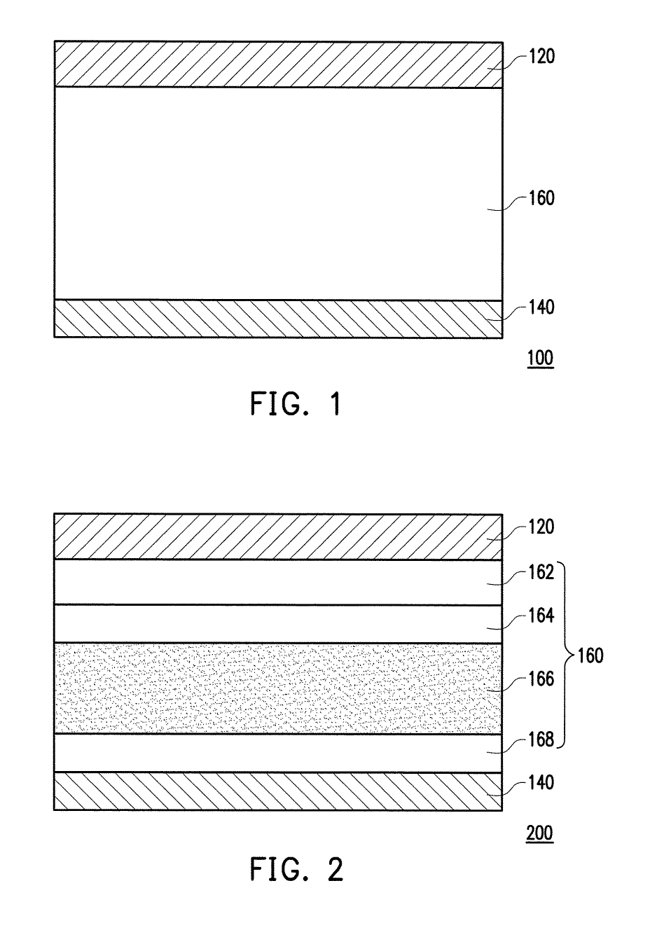

The disclosure further provides an organic electroluminescent device. FIG. 1 is a cross-sectional schematic diagram of an organic electroluminescent device 100 of an embodiment of the disclosure. Referring to FIG. 1, the organic electroluminescent device 100 includes a first electrode layer 120, a second electrode layer 140, and an organic luminescent unit 160. According to the embodiment, the first electrode layer 120 is a transparent electrode material and is, for instance, indium tin oxide (ITO) or other suitable metal oxide materials. The material of the second electrode layer 140 is, for instance, a metal, a transparent conductive material, or other suitable conductive materials. However, the disclosure is not limited thereto. In other embodiments, the first electrode layer 120 is, for instance, a metal, a transparent conductive material, or other suitable conductive materials, and the second electrode layer 140 is, for instance, a transparent electrode material. Specifically, at least one of the first electrode layer 120 and the second electrode layer 140 of the embodiment is a transparent electrode material. In this way, the light emitted by the organic luminescent unit 160 can be emitted through a transparent electrode to make the organic electroluminescent device 100 emit light.

Moreover, FIG. 2 is a cross-sectional schematic diagram of an organic electroluminescent device 200 of another embodiment of the disclosure. Referring to FIG. 2, the organic electroluminescent device 200 is similar to the organic electroluminescent device 100. Therefore, the same or similar devices are represented by the same or similar reference numerals and are not repeated herein. The organic luminescent unit 160 of the organic electroluminescent device 200 can include a hole transport layer 162, an electron blocking layer 164, an organic luminescent layer 166, and an electron transport layer 168.

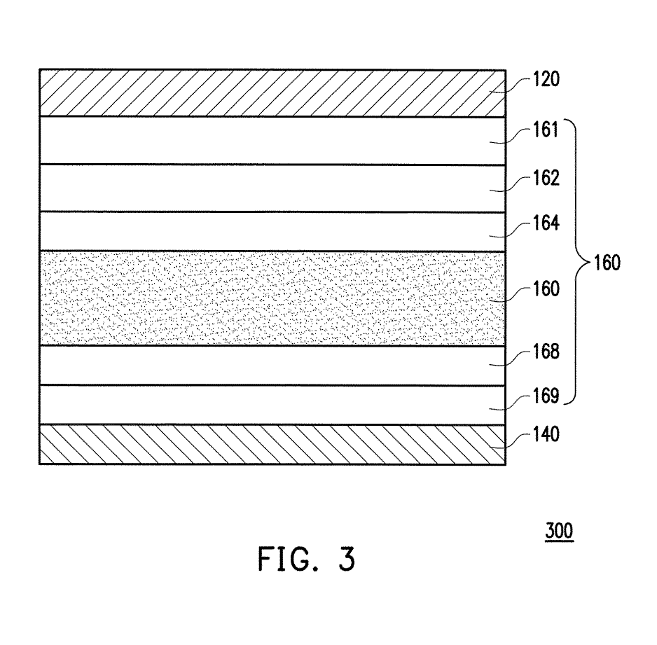

FIG. 3 is a cross-sectional schematic diagram of an organic electroluminescent device of yet another embodiment of the disclosure. Referring to FIG. 3, an organic electroluminescent device 300 is similar to the organic electroluminescent device 100. Therefore, the same or similar devices are represented by the same or similar reference numerals and are not repeated herein. The organic luminescent unit 160 of the organic electroluminescent device 300 can include a hole injection layer 161, a hole transport layer 162, an electron blocking layer 164, an organic luminescent layer 166, an electron transport layer 168, and a hole injection layer 169.

The organic luminescent layer 166 is located between the electron blocking layer 164 and the electron transport layer 168. In the embodiment, the thickness of the organic luminescent layer 166 ranges from, for instance, 5 nm to 60 nm. The organic luminescent layer 166 includes a host material and a guest material. In the embodiment, the host material can include a carbazole derivative shown in formula (1).

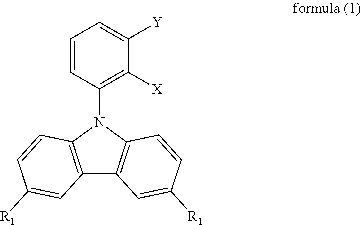

The X in formula (1) can be one of the groups shown in formula (2) to formula (3).

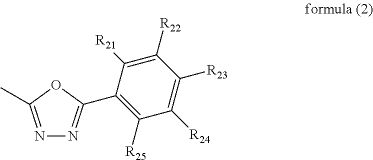

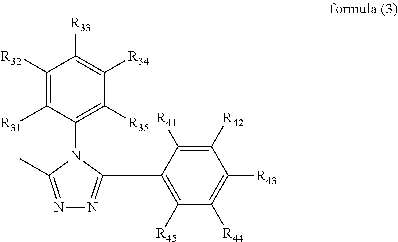

The Y in formula (1) can be R₂ or a group shown in formula (4).

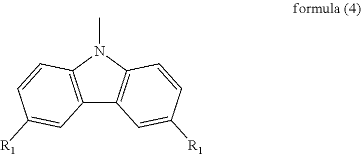

R₁, R₂, R₂₁, R₂₂, R₂₃, R₂₄, R₂₅, R₃₁, R₃₂, R₃₃, R₃₄, R₃₅, R₄₁, R₄₂, R₄₃, and R₄₄ of formula (1) to formula (4) can be selected from one of a hydrogen atom, a fluorine atom, a cyano group, a substituted or non-substituted straight-chain or branched-chain alkyl group, a substituted or non-substituted cycloalkyl group, a substituted or non-substituted straight-chain or branched-chain alkoxy group, a substituted or non-substituted straight-chain or branched-chain thioalkyl group, and a substituted or non-substituted straight-chain or branched-chain alkenyl group; and R₄₅ can be selected from one of a hydrogen atom, a fluorine atom, a cyano group, a substituted or non-substituted straight-chain or branched-chain alkyl group, a substituted or non-substituted cycloalkyl group, a substituted or non-substituted straight-chain or branched-chain alkoxy group, a substituted or non-substituted straight-chain or branched-chain thioalkyl group, a substituted or non-substituted straight-chain or branched-chain alkenyl group, and the group shown in formula (5).

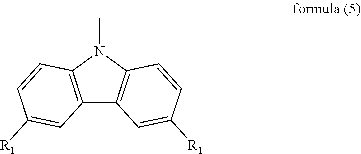

According to an embodiment of the disclosure, the host material of the organic luminescent layer 166 can include one of the carbazole derivatives shown in formula (6) to formula (10). R₁, R₂, R₂₁, R₂₂, R₂₃, R₂₄, R₂₅, R₃₁, R₃₂, R₃₃, R₃₄, R₃₅, R₄₁, R₄₂, R₄₃, and R₄₄ of formula (6) to formula (10) are as defined above.

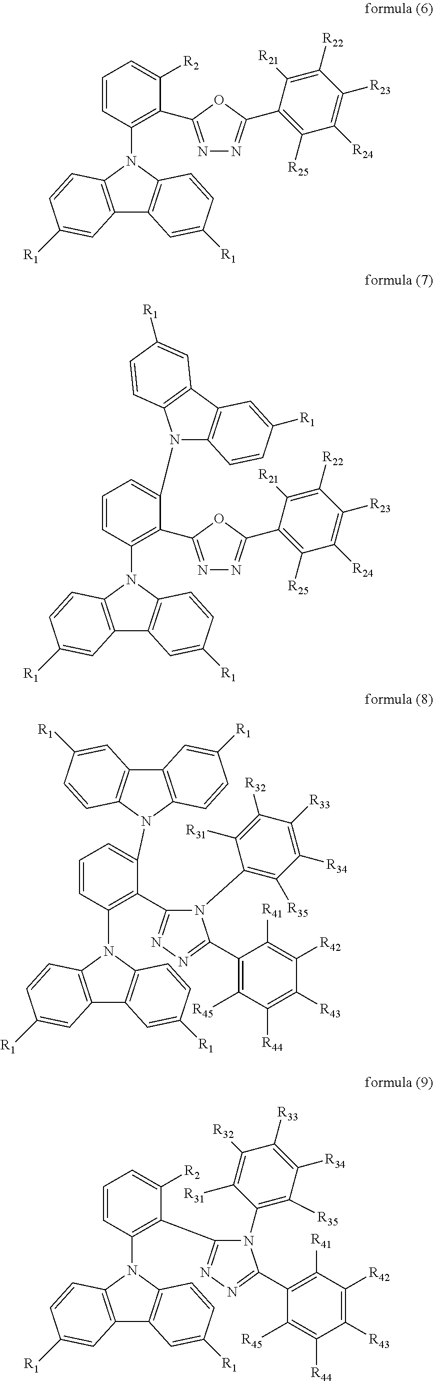

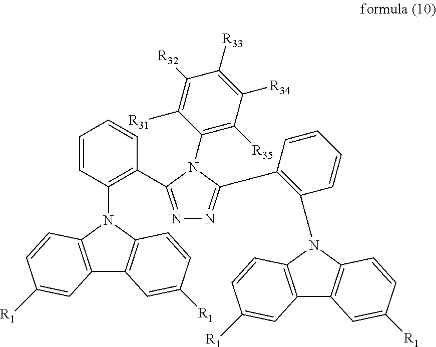

According to an embodiment of the disclosure, the host material of the organic luminescent layer 166 can include one of the carbazole derivatives shown in formula (11) to formula (15).

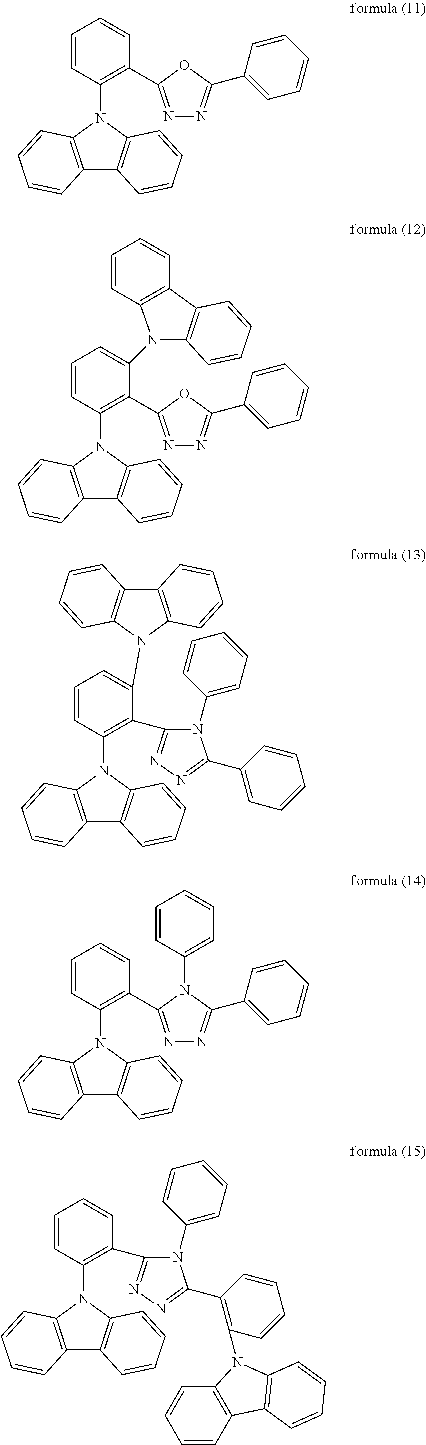

According to the embodiment, the ratio of the host material of any one of formula (1) to formula (15) in the organic luminescent layer 166 is, for instance, 60 volume % to 99.5 volume %.

In the embodiment, the guest material is, for instance, one of the compounds shown in formula (16) to formula (18). However, the disclosure is not limited thereto.

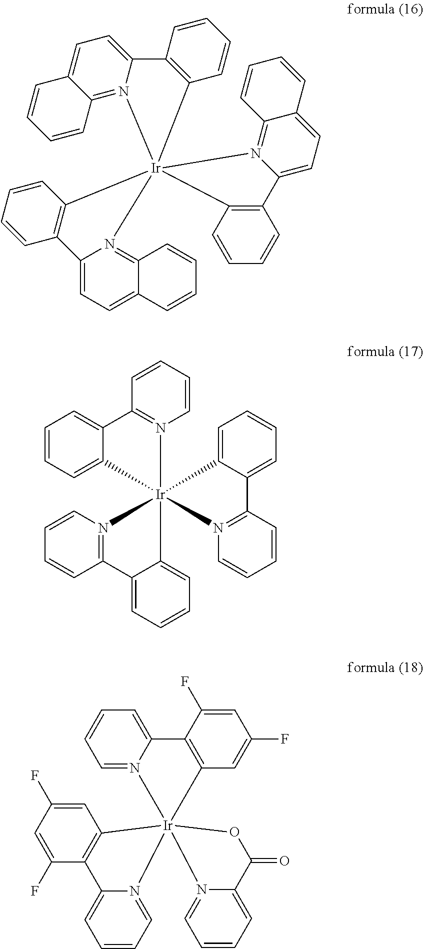

According to the embodiment, the ratio of the guest material in the organic luminescent layer 166 is, for instance, 0.5 volume % to 40 volume %.

Referring to FIG. 3, the hole transport layer 162 of the organic electroluminescent device 300 is located between the hole injection layer 161 and the electron blocking layer 164. The material of the hole transport layer 162 is, for instance, a known material such as N,N′-diphenyl-N,N′-bis(1-naphthyl)(1,1′-biphenyl)-4,4′diamine (NPB) or N,N′-diphenyl-N,N′-bis(3-methylphenyl)-1,1′-biphenyl-4,4′-diamine (TPD). In the embodiment, the thickness of the hole transport layer 162 ranges from, for instance, 0 nm to 100 nm. The hole transport layer 162 can increase the injection rate of holes from the first electrode layer 120 to the organic luminescent layer 166 and reduce the driving voltage of the organic electroluminescent device 300 at the same time.

Referring to FIG. 3, the electron blocking layer 164 is located between the hole transport layer 162 and the organic luminescent layer 166. The material of the electron blocking layer 164 is, for instance, mCP or other materials having low electron affinity. In the embodiment, the thickness of the electron blocking layer 164 ranges from, for instance, 0 nm to 30 nm. The electron blocking layer 164 can further increase the transport rate of holes from the hole transport layer 162 to the organic luminescent layer 166 and confine holes and electrons to recombine and emit in the organic luminescent layer 166.

Referring to FIG. 3, the electron transport layer 168 is located between the organic luminescent layer 166 and the electron injection layer 169. The material of the electron transport layer 168 is, for instance, a metal complex such as AlQ or BeBq₂ or a heterocyclic compound such as PBD, TAZ, or TPBI. In the embodiment, the thickness of the electron transport layer 168 ranges from, for instance, 0 nm to 100 nm. The electron transport layer 168 can facilitate the transfer of electrons from the second electrode layer 140 to the organic luminescent layer 166 to increase the transport rate of the electron.

A plurality of synthesis examples are listed below to describe the production process of the carbazole derivatives of formula (11) to formula (15) of the disclosure in detail.

### Synthesis Example 1: Synthesis of the Compound of Formula (11)

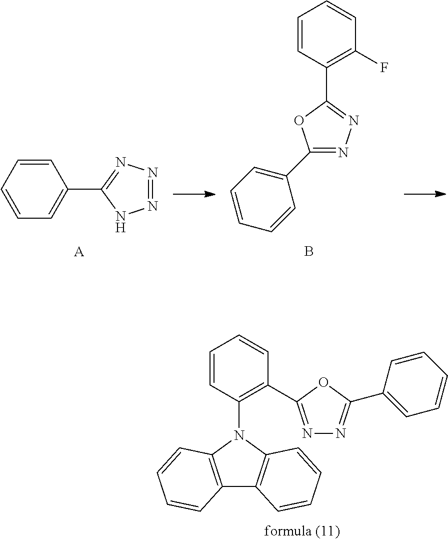

First, sodium azide (20.00 grams, 307.9 millimoles), ammonium chloride (16.86 grams, 314.5 millimoles), and a magnet were put in a 500 milliliter two-neck bottle. After a condenser was installed, benzonitrile (15.7 milliliters, 152.3 millimoles) and dimethyl formamide (160 milliliters) were injected under a nitrogen system, and the reactants were refluxed at 125° C. for 24 hours to remove the solvent through distillation under reduced pressure. Then, deionized water (80 milliliters) was added and the reactants were stirred. After a large amount of white precipitate was observed, hydrochloric acid (12N, 3 milliliters) was slowly added dropwise to quench the remaining sodium azide. At this point, a large amount of highly toxic hydrazoic acid was produced. The reactants were stirred at room temperature for 24 hours. After the volatilization of the hydrazoic acid was exhausted, a white solid was collected through suction filtration and by using water as a washing fluid. Then, ethanol was used to perform recrystallization. Next, using acetone as a washing fluid, the solid was collected through suction filtration. Lastly, the solvent was removed using a vacuum system to obtain a white needle-like solid (compound A, 20.01 grams, 89% yield).

Next, the compound A (9.60 grams, 66.67 millimoles) and a magnet were placed in a 250 milliliter two-neck bottle. Then, a condenser was installed, and dry pyridine (100 milliliters) and 2-fluorobenzoyl chloride (7.7 milliliters, 56.88 millimoles) were injected under a nitrogen system. After the reactants were refluxed at 90° C. for 24 hours, precipitation was performed using dilute hydrochloric acid to observe the precipitation of a light brown solid. The solid was collected through suction filtration and recrystallization was performed using acetone. Using methanol as a washing fluid, the solid was collected through suction filtration. Lastly, after the solvent was removed using a vacuum system, a white flaky crystal (compound B, 12.83 grams, 86% yield) was obtained.

Then, caesium carbonate (10.6 grams, 32.53 millimoles), carbazole (5.00 grams, 29.94 millimoles), and a magnet were placed in a 250 milliliter two-neck bottle. A condenser was installed, and dimethyl sulfoxide (84 milliliters) was injected under an argon system. After the reactants were stirred at normal temperature for 30 minutes, the compound B (7.10 grams, 29.55 millimoles) was added. After the reactants were reacted at 160° C. for 24 hours, the precipitation of a solid was observed on the wall of the bottle. After the solid was precipitated using dilute hydrochloric acid, the solid was collected through suction filtration and by using water as a washing fluid. Recrystallization was performed using dichloromethane and acetone. Next, using acetone as a washing fluid, the solid was collected through suction filtration. Lastly, after the solvent was removed using a vacuum system, a white crystalline compound of formula (11) (10.00 grams, 87% yield) was obtained.

### Synthesis Example 2: Synthesis of the Compound of Formula (14)

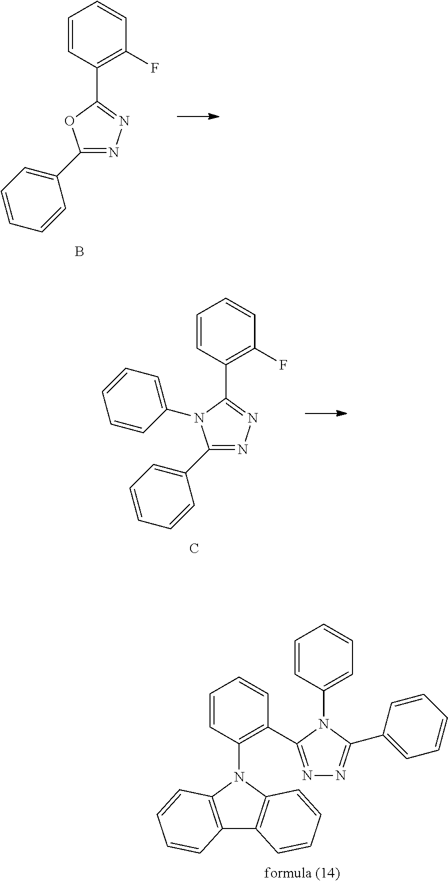

Aluminium chloride (1.83 grams, 13.72 millimoles), aniline (5.1 milliliters, 55.91 millimoles), and a magnet were placed in a 50 milliliter wide mouth two-neck bottle. A condenser was installed and the bottle was heated to 160° C. under an argon system. Then, after the reactants were refluxed for 2.5 hours, the temperature was returned to 160° C. The compound B (5.00 grams, 20.81 millimoles) was added and dry N-methylpyrrolidinone (5.8 milliliters) was injected. After the reactants were refluxed at 202° C. for 24 hours, the temperature was returned to 202° C. Precipitation was performed using dilute hydrochloric acid, and the solid was collected through suction filtration and by using water as a washing fluid. Recrystallization was performed using methanol, and the solid was collected through suction filtration and by using acetone as a washing fluid. Lastly, the solvent was removed using a vacuum system to obtain a white solid (compound C, 5.92 grams, 90% yield).

Then, caesium carbonate (3.33 grams, 10.21 millimoles), carbazole (1.55 grams, 9.28 millimoles), and a magnet were placed in a 50 milliliter two-neck bottle. A condenser was installed, and N-methylpyrrolidinone (25 milliliters) was injected under an argon system. After the reactants were stirred at normal temperature for 30 minutes, the compound C (3.08 grams, 9.77 millimoles) was added. After reacting at 210° C. for 72 hours, the reactants were poured into water to remove N-methylpyrrolidinone. At this point, a creamy yellow and misty aqueous solution was observed. Extraction was performed using ether, and the water layer was changed into a black clarified aqueous solution. After water was removed using an organic layer and anhydrous magnesium, the solvent was removed using a rotary evaporator, and recrystallization was performed using dichloromethane and acetone. The solid was collected through suction filtration, and after the solvent was removed using a vacuum system, a white solid compound of formula (14) (3.16 grams, 70% yield) was obtained.

### Synthesis Example 3: Synthesis of the Compound of Formula (12)

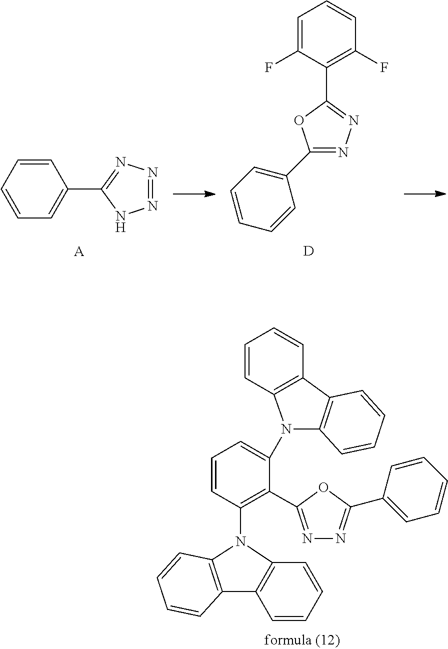

The compound A (4.55 grams, 31.13 millimoles) and a magnet were placed in a 100 milliliter two-neck bottle. A condenser was installed, and dry pyridine (30 milliliters) and 2,6-difluorobenzoyl chloride (3.56 milliliters, 28.32 millimoles) were injected under a nitrogen system. After the reactants were refluxed at 90° C. for 24 hours, precipitation was performed using dilute hydrochloric acid to observe the precipitation of a light brown solid. The solid was collected through suction filtration, and recrystallization was performed using acetone, and then the solid was collected through suction filtration and by using methanol as a washing fluid. Lastly, the solvent was removed using a vacuum system, and a white needle-like crystal (compound D, 5.86 grams, 80% yield) was obtained.

Then, caesium carbonate (1.87 grams, 5.74 millimoles), carbazole (0.8828 grams, 5.29 millimoles), and a magnet were placed in a 25 milliliter two-neck bottle. A condenser was installed, and dimethyl sulfoxide (7.5 milliliters) was injected under an argon system. After the reactants were stirred at normal temperature for 30 minutes, the compound D (0.65 grams, 2.52 millimoles) was added. After the reactants were reacted at 160° C. for 24 hours, a solid was precipitated on the wall of the bottle. After the solid was precipitated using dilute hydrochloric acid, the solid was collected through suction filtration and by using water as a washing fluid. Recrystallization was performed using dichloromethane and acetone, and the solid was collected through suction filtration and by using acetone as a washing fluid. Lastly, the solvent was removed using a vacuum system, and a white crystalline compound of formula (12) (1.38 grams, 92.62% yield) was obtained.

### Synthesis Example 4: Synthesis of the Compound of Formula (13)

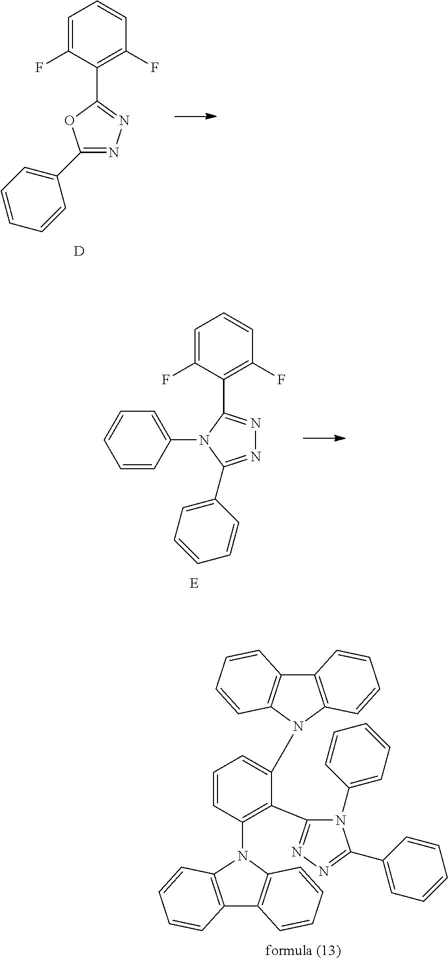

Aluminium chloride (0.68 grams, 5.10 millimoles), aniline (1.86 milliliters, 20.40 millimoles), and a magnet were placed in a 5 milliliter two-neck bottle. A condenser was installed and the bottle was heated to 160° C. under an argon system. Then, after the reactants were refluxed for 2.5 hours, the temperature was returned to 160° C. The compound D (2.00 grams, 7.75 millimoles) was added and dry N-methylpyrrolidinone (1.55 milliliters) was injected. After the reactants were refluxed at 202° C. for 24 hours, the temperature was returned to 202° C. Precipitation was performed using dilute hydrochloric acid, and the solid was collected through suction filtration and by using water as a washing fluid. Recrystallization was performed using methanol, and the solid was collected through suction filtration and by using acetone as a washing fluid. Lastly, the solvent was removed using a vacuum system to obtain a white solid (compound E, 2.15 grams, 83% yield).

Then, caesium carbonate (3.07 grams, 9.43 millimoles), carbazole (1.45 grams, 8.68 millimoles), and a magnet were placed in a 25 milliliter two-neck bottle. A condenser was installed, and dimethyl sulfoxide (12 milliliters) was injected under an argon system. After the reactants were stirred at normal temperature for 30 minutes, the compound D (1.40 grams, 4.20 millimoles) was added. After the reactants were reacted at 160° C. for 72 hours, a solid was precipitated on the wall of the bottle. After the solid was precipitated using dilute hydrochloric acid, the solid was collected through suction filtration and by using water as a washing fluid. Recrystallization was performed using dichloromethane and acetone, and the solid was collected through suction filtration and by using acetone as a washing fluid. Lastly, the solvent was removed using a vacuum system, and a white crystalline compound of formula (13) (2.21 grams, 79% yield) was obtained.

### Synthesis Example 5: Synthesis of the Compound of Formula (15)

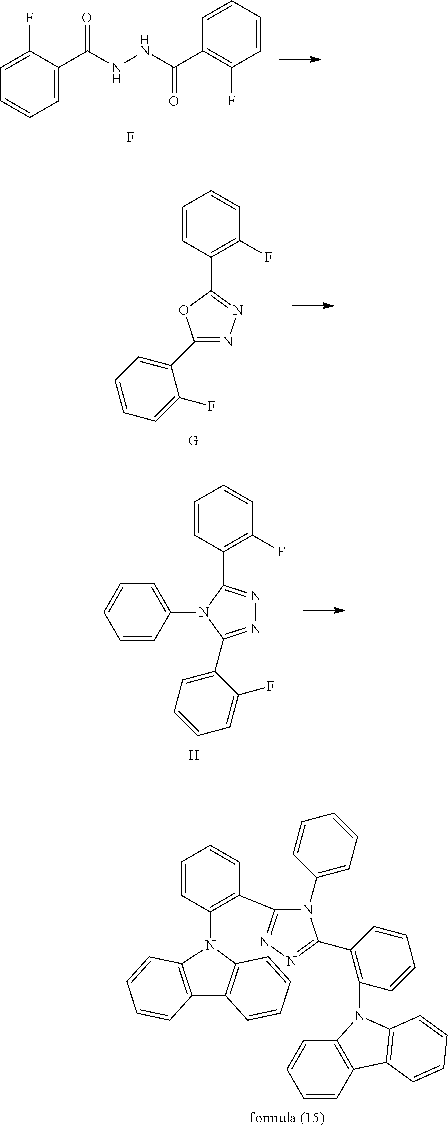

A magnet was placed in a 100 milliliter two-neck bottle, and tetrahydrofuran (50 milliliters) and 64% hydrazine (0.7 milliliters, 14 millimoles) were injected into the bottle under a nitrogen system. After stirring for 20 minutes in a water bath, 2-fluorobenzoyl chloride (3.8 milliliters, 28.07 millimoles) was injected into the bottle. After stirring for 24 hours at room temperature, the solvent and excess hydrazine were removed through distillation under normal pressure. After the reactants were hot washed with alcohol, the solid was collected through suction filtration. Lastly, the solvent was removed using a vacuum system to obtain a white crude product (compound F, 1.9 grams, 44% yield).

Next, the compound F (2 grams, 7.24 millimoles) and a magnet were placed in a 50 milliliter single-neck bottle. Then, thionyl chloride (15 milliliters) and several drops of dimethylformamide (DMF) were added, and a condenser was installed. The reactants were refluxed at 75° C. for 24 hours and then the temperature was returned to 75° C. The reactants were slowly added into cold water dropwise to quench the thionyl chloride. A solid was precipitated at this point. Using water as a washing fluid, the solid was collected through suction filtration. Recrystallization was performed using acetone, and the solid was collected through suction filtration and by using acetone as a washing fluid. Lastly, the solvent was removed using a vacuum system, and a white crystal (compound G, 1.7 grams, 91% yield) was obtained.

Then, aluminium chloride (0.51 grams, 3.82 millimoles), aniline (1.4 milliliters, 15.33 millimoles), and a magnet were placed in a 5 milliliter two-neck bottle. A condenser was installed and the bottle was heated to 160° C. under an argon system. Then, after the reactants were refluxed for 2.5 hours, the temperature was returned to 160° C. The compound G (1.50 grams, 5.80 millimoles) was added and dry N-methylpyrrolidinone (1.0 milliliter) was injected. After the reactants were refluxed at 202° C. for 24 hours, the temperature was returned to 202° C. Precipitation was performed using dilute hydrochloric acid, and the solid was collected through suction filtration and by using water as a washing fluid. Recrystallization was performed using methanol, and the solid was collected through suction filtration and by using acetone as a washing fluid. Lastly, the solvent was removed using a vacuum system to obtain a white solid (compound H, 1.64 grams, 84% yield).

Then, caesium carbonate (2.86 grams, 8.78 millimoles), carbazole (1.35 grains, 8.10 millimoles), and a magnet were placed in a 25 milliliter two-neck bottle. A condenser was installed, and N-methylpyrrolidinone (11 milliliters) was injected under an argon system. After the reactants were stirred at normal temperature for 30 minutes, the compound H (1.35 grams, 4.05 millimoles) was added. After the reactants were reacted at 210° C. for 24 hours, the solid was precipitated using dilute hydrochloric acid. Then, the solid was collected through suction filtration and by using water as a washing fluid. Recrystallization was performed using dichloromethane and acetone, and the solid was collected through suction filtration and by using acetone as a washing fluid. Lastly, the solvent was removed using a vacuum system, and a white crystalline compound of formula (15) (1.9 grams, 70% yield) was obtained.

### [Evaluation Methods of Host Material]

The host material of the embodiments of the disclosure includes the compounds synthesized according to synthesis example 1 to synthesis example 5 (i.e., the carbazole derivatives of formula (11) to formula (15)). The evaluation methods of the host material include respectively measuring the compounds for triplet state energy level (E_{T}), glass transition temperature (T_{g}), thermal degradation temperature (T_{d}), highest occupied molecular orbital energy level (HOMO), and lowest unoccupied molecular orbital energy level (LUMO). Moreover, the known host material mCP is used as a comparative example. The glass transition temperature (T_{g}) is measured and obtained with a differential scanning calorimeter (DSC), and the thermal degradation temperature was obtained by measuring the temperature of a material at a loss of 5 weight % using a themiogravimetric analyzer (TGA). The results are shown in Table

TABLE 1

| | E_{T} | T_{g} | T_{d} | | |
|---|---|---|---|---|---|
| | (eV) | (° C.) | (° C.) | HOMO(eV) | LUMO(eV) |
| Synthesis | 2.80 | — | 278 | 5.7 | 2.7 |
| example 1: | | | | | |
| formula (11) | | | | | |
| Synthesis | 3.00 | 98 | 394 | 5.8 | 2.7 |
| example 2: | | | | | |
| formula (14) | | | | | |
| Synthesis | 3.09 | 87 | 349 | 5.7 | 2.3 |
| example 3: | | | | | |
| formula (12) | | | | | |
| Synthesis | 3.07 | — | 365 | 5.7 | 2.3 |
| example 4: | | | | | |
| formula (13) | | | | | |
| Synthesis | 3.09 | — | 353 | 5.7 | 2.3 |
| example 5: | | | | | |
| formula (15) | | | | | |
| Comparative | 2.90 | 55 | — | 5.8 | 2.3 |
| example | | | | | |

It should be mentioned that, Flrpic is used as the guest material here as an example. Referring to Table 1, although the triplet state energy level (2.9 eV) of the comparative example is higher than the triplet state energy level (2.7 eV) of the Flrpic, the glass transition temperature thereof is only 55° C., and therefore the thermal stability thereof is poor. The triplet state energy level of each of synthesis example 1 to synthesis example 5 is equal to the triplet state energy level of the guest material FIrpic, and the glass transition temperature of each of synthesis example 2 and synthesis example 3 is higher than the glass transition temperature of the comparative example. Therefore, the compounds of synthesis examples 1 to 5 are suitable for the host luminescent material in the organic luminescent layer.

The application of the carbazole derivatives of formula (11) to formula (15) to the organic electroluminescent device of the host material is respectively described in the following with a plurality of examples. The luminous efficiency of the luminescent device is also verified.

### Example 1: Organic Electroluminescent Device Using the Carbazole Derivative of Formula (11) as Host Material

In the present example, the material of the first electrode layer of the organic electroluminescent device is ITO. The material of the second electrode layer is aluminium and the thickness thereof is 120 nm. The material of the hole transport layer is NPB and the thickness thereof is 50 nm. The material of the electron blocking layer is mCP and the thickness thereof is 10 nm. The material of the electron transport layer is TAZ and the thickness thereof is 40 nm. The host material of the organic luminescent layer is the carbazole derivative of formula (11), and the doping ratio thereof is 91 volume %. The host material is respectively used with the compounds of formula (16) to formula (18) (i.e., the known Ir(2-phq)₃, Ir(ppy)₃, and FIrpic) used as the guest material, and the doping ratio of the guest material is 9 volume %. The thickness of the organic luminescent layer is 30 nm. The organic electroluminescent device of the present example is completed by forming each film layer above through vapor deposition, and then driving voltage (V) at an injection current density of 40 mA/cm², external quantum efficiency (EQE) (%), maximum current efficiency (cd/A), maximum power efficiency (lm/W), and maximum brightness (cd/m²) at 12V thereof are evaluated. The evaluation results are shown in Table 2.

TABLE 2

| | Driving | Bright- | Current | Power | |
|---|---|---|---|---|---|
| | voltage | ness | efficiency | efficiency | |
| Device | (V) | (cd/m²) | (cd/A) | (lm/W) | EQE(%) |
| formula (11): | 10.68 | 11340 | 28.79 | 17.33 | 13.27 |
| Ir(2-phq)₃ | | | | | |
| (magenta light) | | | | | |
| formula (11): | 10.41 | 30390 | 51.23 | 28.04 | 14.92 |
| Ir(ppy)₃ | | | | | |
| (green light) | | | | | |
| formula (11): | 8.72 | 5399 | 23.10 | 15.97 | 8.66 |
| FIrpic (sky | | | | | |
| blue light) | | | | | |

### Example 2: Organic Electroluminescent Device Using the Carbazole Derivative of Formula (12) as Host Material

The present example is similar to example 1, and the only difference is that the carbazole derivative of formula (12) is used for the host material of the organic luminescent layer. The evaluation results are shown in Table 3.

TABLE 3

| | Driving | Bright- | Current | Power | |
|---|---|---|---|---|---|
| | voltage | ness | efficiency | efficiency | |
| Device | (V) | (cd/m²) | (cd/A) | (lm/W) | EQE(%) |
| formula (12): | 10.84 | 15210 | 36.35 | 23.11 | 15.10 |
| Ir(2-phq)₃ | | | | | |
| (magenta light) | | | | | |
| formula (12): | 10.52 | 33760 | 77.70 | 51.40 | 19.03 |
| Ir(ppy)₃ | | | | | |
| (green light) | | | | | |
| formula (12): | 9.40 | 8695 | 40.60 | 31.02 | 16.59 |
| FIrpic (sky | | | | | |
| blue light) | | | | | |

### Example 3: Organic Electroluminescent Device Using the Carbazole Derivative of Formula (14) as Host Material

In the present example, the material of the first electrode layer of the organic electroluminescent device is ITO. The material of the second electrode layer is aluminium and the thickness thereof is 120 nm. The material of the hole transport layer is NPB and the thickness thereof is 50 nm. The material of the electron blocking layer is mCP and the thickness thereof is 10 nm. The material of the electron transport layer is TAZ. The host material of the organic luminescent layer is the carbazole derivative of formula (14), and the doping ratio thereof is 91 volume %. The host material is respectively used with the compounds of formula (17) and formula (18) (i.e., the known Ir(ppy)₃ and FIrpic) used as the guest material, and the doping ratio of the guest material is 9 volume %. When the guest material used is the compound of formula (17), the thickness of the organic luminescent layer is 30 nm, and the thickness of the electron transport layer is 40 nm. When the guest material used is the compound of formula (18), the thickness of the organic luminescent layer is 40 nm, and the thickness of the electron transport layer is 47 nm. The organic electroluminescent device of the present example is completed by forming each film layer above through vapor deposition, and the evaluation results thereof are listed in Table 4.

TABLE 4

| | Driving | Bright- | Current | Power | |
|---|---|---|---|---|---|
| | voltage | ness | efficiency | efficiency | |
| Device | (V) | (cd/m²) | (cd/A) | (lm/W) | EQE(%) |
| formula (14): | 12 | 35360 | 78.34 | 70.32 | 21.82 |
| Ir(ppy)₃ | | | | | |
| (green light) | | | | | |
| formula (14): | 9.46 | 9560 | 52.11 | 46.14 | 22.94 |
| FIrpic (sky | | | | | |
| blue light) | | | | | |

### Example 4: Organic Electroluminescent Device Using the Carbazole Derivative of Formula (13) as Host Material

In the present example, the material of the first electrode layer of the organic electroluminescent device is ITO. The material of the second electrode layer is aluminium and the thickness thereof is 120 nm. The material of the hole transport layer is NPB and the thickness thereof is 50 nm. The material of the electron blocking layer is mCP and the thickness thereof is 10 nm. The material of the electron transport layer is TAZ. The host material of the organic luminescent layer is the carbazole derivative of formula (13), and the host material is respectively used with the compounds of formula (17) and formula (18) (i.e., the known Ir(ppy)₃ and FIrpic) used as the guest material. In particular, when the guest material used is the compound of formula (17), the doping ratio of the guest material is 12 volume %, the thickness of the organic luminescent layer is 30 nm, and the thickness of the electron transport layer is 40 nm. When the guest material used is the compound of formula (18), the doping ratio of the guest material is 16.8 volume %, the thickness of the organic luminescent layer is 40 nm, and the thickness of the electron transport layer is 60 nm. The organic electroluminescent device of the present example is completed by forming each film layer above through vapor deposition, and the evaluation results thereof are listed in Table 5.

TABLE 5

| | Driving | Bright- | Current | Power | |
|---|---|---|---|---|---|
| | voltage | ness | efficiency | efficiency | |
| Device | (V) | (cd/m²) | (cd/A) | (lm/W) | EQE(%) |
| formula (13): | 9.78 | 40040 | 79.87 | 62.84 | 21.94 |
| Ir(ppy)₃ | | | | | |
| (green light) | | | | | |
| formula (13): | 10.59 | 15200 | 51.36 | 46.10 | 23.29 |
| FIrpic (sky | | | | | |
| blue light) | | | | | |

### Example 5: Organic Electroluminescent Device Using the Carbazole Derivative of Formula (15) as Host Material

In the present example, the material of the first electrode layer of the organic electroluminescent device is ITO. The material of the second electrode layer is aluminium and the thickness thereof is 120 nm. The material of the hole transport layer is NPB and the thickness thereof is 50 nm. The material of the electron blocking layer is mCP and the thickness thereof is 10 nm. The material of the electron transport layer is TAZ and the thickness thereof is 40 nm. The host material of the organic luminescent layer is the carbazole derivative of formula (15), and the host material is used with the compound of formula (18) (i.e., the known FIrpic) used as the guest material, wherein the doping ratio of the guest material is 9 volume % and the thickness of the organic luminescent layer is 30 nm. The organic electroluminescent device of the present example is completed by forming each film layer above through vapor deposition, and the evaluation results thereof are listed in Table 6.

TABLE 6

| | Driving | Bright- | Current | Power | |
|---|---|---|---|---|---|
| | voltage | ness | efficiency | efficiency | |
| Device | (V) | (cd/m²) | (cd/A) | (lm/W) | EQE(%) |
| formula (15): | 9.29 | 16410 | 39.36 | 36.11 | 17.03 |
| FIrpic (sky | | | | | |
| blue light) | | | | | |

It can be known from the results of Table 2 to Table 6 that, the organic electroluminescent device of each of example 1 to example 5 not only can have a low driving voltage, but can also have good current efficiency, power efficiency, and external quantum efficiency. Therefore, by including the bipolar carrier transport properties of the host material of the disclosure, the transport rate of electrons and holes can be increased. As a result, the organic electroluminescent device of each of example 1 to example 5 can be operated without a high driving voltage. It should be mentioned that, the external quantum efficiency of the organic electroluminescent device of each of example 1 to example 5 is higher than the external quantum efficiency (8%) of the organic electroluminescent device using mCP as the host material. Therefore, the host material of each of example 1 to example 5 has a higher triplet state energy level, thus facilitating the reduction of the phenomenon of reverse energy transfer. As a result, the luminescent efficiency of the organic electroluminescent device can be increased.

Based on the above, when the organic luminescent material of the disclosure is used as the host material, the host material has a higher triplet state energy level. Therefore, the energy of the guest material is not readily returned to the host material and energy loss can thereby be reduced. Moreover, the carbazole derivative of the host material of the disclosure is formed by an electron-accepting group and an electron-donating group, and therefore has good bipolar carrier transport properties. As a result, the driving voltage of the organic electroluminescent device can further be reduced. Moreover, the organic luminescent material of the disclosure containing the carbazole derivative has the characteristic of a high molecular weight and can have a higher glass transition temperature. In other words, the organic luminescent material of the disclosure can have good thermal stability and is suitable for the organic electroluminescent device.

Although the disclosure has been described with reference to the above embodiments, it will be apparent to one of the ordinary skill in the art that modifications to the described embodiments may be made without departing from the spirit of the disclosure. Accordingly, the scope of the disclosure is defined by the attached claims not by the above detailed descriptions.

What is claimed is:

1. A carbazole derivative shown in formula (9): 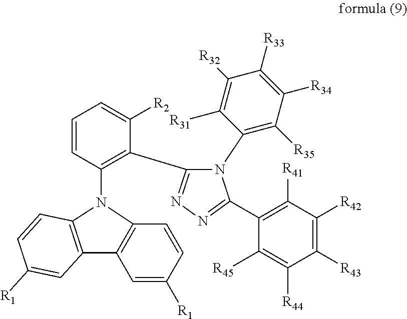
wherein, R₁, R₂, R₃₁, R₃₂, R₃₃, R₃₄, R₃₅, R₄₁, R₄₂, R₄₃, and R₄₄ are independently selected from one of a hydrogen atom, a fluorine atom, a cyano group, a substituted or non-substituted straight-chain or branched-chain alkyl group, a substituted or non-substituted cycloalkyl group, a substituted or non-substituted straight-chain or branched-chain alkoxy group, a substituted or non-substituted straight-chain or branched-chain thioalkyl group, and a substituted or non-substituted straight-chain or branched-chain alkenyl group, and
R₄₅ is independently selected from one of a fluorine atom, a cyano group, a substituted or non-substituted straight-chain or branched-chain alkyl group, a substituted or non-substituted cycloalkyl group, a substituted or non-substituted straight-chain or branched-chain alkoxy group, a substituted or non-substituted straight-chain or branched-chain thioalkyl group, a substituted or non-substituted straight-chain or branched-chain alkenyl group, and a group shown in formula (5):

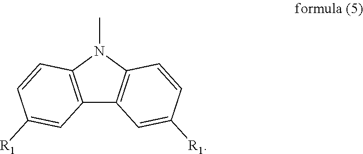

2. The carbazole derivative as claimed in claim 1, wherein the carbazole derivative is shown in formula (10):

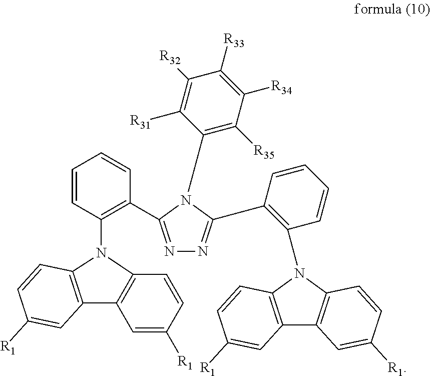

3. The carbazole derivative as claimed in claim 1, wherein the carbazole derivative is shown in formula (15):

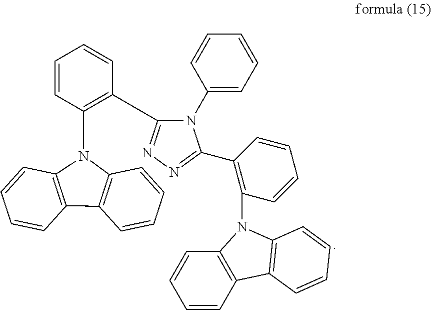 [SMILES: c1ccc(-n2c(-c3ccccc3-n3c4ccccc4c4ccccc43)nnc2-c2ccccc2-n2c3ccccc3c3ccccc32)cc1]

4. An organic electroluminescent device, comprising:
a first electrode layer;
a second electrode layer; and
an organic luminescent unit disposed between the first electrode layer and the second electrode layer, wherein the organic luminescent unit comprises the carbazole derivative as claimed in claim 1.

5. The organic electroluminescent device of claim 4, wherein the organic luminescent unit further comprises a hole injection layer, a hole transport layer, an electron blocking layer, an electron injection layer, an electron transport layer, or a combination thereof.

6. An organic electroluminescent device, comprising:
a first electrode layer;
a second electrode layer; and
an organic luminescent unit disposed between the first electrode layer and the second electrode layer, wherein the organic luminescent unit comprises an organic luminescent layer, and the organic luminescent layer comprises a host material and a guest material, wherein the host material comprises the carbazole derivative as claimed in claim 1.

7. The organic electroluminescent device of claim 6, wherein a ratio of the host material in the organic luminescent layer is 60 volume % to 99.5 volume %.

8. The organic electroluminescent device of claim 6, wherein the guest material comprises one of the compounds shown in formula (16) to formula (18):

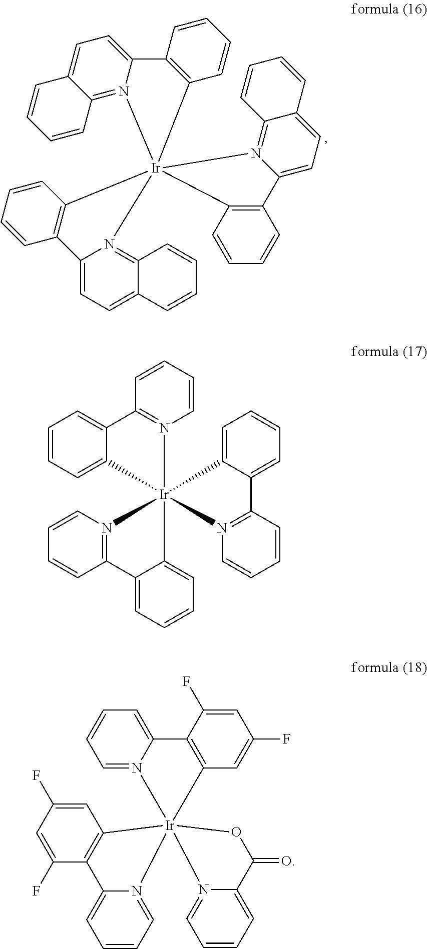

9. The organic electroluminescent device of claim 6, wherein a ratio of the guest material in the organic luminescent layer is 0.5 volume % to 40 volume %.

10. The organic electroluminescent device of claim 6, wherein at least one of the first electrode layer and the second electrode layer is a transparent electrode material.
